(19) United States
(12) Patent Application Publication
(10) Pub. No.: US 2016/0220099 A1
(43) Pub. Date: Aug. 4, 2016

(54) ENDOSCOPE HAVING AN ADJUSTABLE VIEWING DIRECTION

(71) Applicant: OLYMPUS WINTER & IBE GMBH, Hamburg (DE)

(72) Inventors: Peter SCHOUWINK, Ahrensburg (DE); Regina ORZEKOWSKY-SCHROEDER, Luebeck (DE)

(73) Assignee: OLYMPUS WINTER & IBE GMBH, Hamburg (DE)

(21) Appl. No.: 15/094,156

(22) Filed: Apr. 8, 2016

Related U.S. Application Data
(63) Continuation of application No. PCT/EP2014/002669, filed on Oct. 1, 2014.

(30) Foreign Application Priority Data
Oct. 16, 2013 (DE) 102013220945.2

(51) Int. Cl. A61B 1/00; A61B 1/05

(52) U.S. Cl. CPC A61B 1/00183; A61B 1/0019; A61B 1/05; A61B 1/00078; A61B 1/00096

(57) ABSTRACT

An endoscope having an adjustable viewing direction at a viewing angle, the endoscope including: an endoscope shaft having a longitudinal axis, wherein the endoscope shaft has a distal viewing window, a movable optical device for capturing objects provided in the endoscope shaft, and a focusing optical unit arranged between a distal viewing window and the movable optical device, wherein a focal distance of the focusing optical unit is changed in dependence on the viewing angle.

### CROSS-REFERENCE TO RELATED APPLICATION

The present application is a continuation of PCT/EP2014/002669 filed on Oct. 1, 2014, which is based upon and claims the benefit to DE 10 2013 220 945.2 filed on Oct. 16, 2013, the entire contents of each of which are incorporated herein by reference.

### BACKGROUND

1. Field

The present application relates to an endoscope, in particular a video endoscope, having an adjustable viewing direction at a viewing angle, said endoscope comprising an endoscope shaft having a longitudinal axis, wherein the endoscope shaft has a distal viewing window, wherein a movable optical device for capturing objects is provided in the endoscope shaft, preferably at the proximal end.

2. Prior Art

Endoscopes, and in particular video endoscopes, in which the light of a field of operation which is incident at a distal tip of an endoscope shaft of the endoscope is directed through an optical system to one or more image sensors, are known in different embodiments. Thus, endoscopes are available with a forward viewing direction, a so-called 0° viewing direction, or endoscopes are available with a lateral viewing direction, which for example have a lateral viewing direction of 30°, 45°, 70° or the like deviating from the 0° viewing direction. In this case, the aforementioned number of degrees is understood as the angle between the central viewing axis and the longitudinal axis of the endoscope shaft. Moreover, endoscopes or respectively video endoscopes are available with an adjustable lateral viewing direction, in which the viewing angle, i.e. the deviation from the view straight ahead, is adjustable.

During an adjustment of the viewing angle, therefore, the deviation from the view straight ahead is altered, in particular relative to the longitudinal axis of the endoscope shaft.

Moreover, an optical system for a video endoscope is disclosed in EP 2 369 395 A1 in which the viewing angle is altered by a prism of a prism unit with three prisms being rotated about an axis of rotation which is located perpendicular or respectively transversely to the longitudinal axis of the endoscope shaft. The two other prisms which, together with the first prism, define the optical beam path, are not rotated therewith so that the reflective surface of the first prism which is rotated, rotates relative to the corresponding reflective surface of the second prism.

A further endoscope with a variable viewing direction is disclosed in DE 10 2010 028 147 A1.

### SUMMARY

Proceeding from this prior art, an object is to provide an endoscope having an adjustable viewing direction in which, when adjusting the viewing angle, the capture of desired objects and improved focusing of the captured object images are permitted in a simple manner, for different viewing angle settings.

Such object can be achieved by an endoscope, such as a video endoscope, having an adjustable viewing direction at a viewing angle, said endoscope comprising an endoscope shaft having a longitudinal axis, wherein the endoscope shaft has a distal viewing window, wherein a movable optical device for capturing objects is provided in the endoscope shaft, said endoscope being developed in that a focusing optical unit is arranged between the distal viewing window and the movable optical device, wherein the focal distance of the focusing optical unit can be changed or is changed in dependence on the viewing angle.

The exemplary embodiments disclosed herein are based on the idea that, by the use of a focusing optical unit having a plurality of focal distances, for example a gradient index lens or optionally a plurality of gradient index lenses or a diffractive and/or refractive optical unit, between the distal viewing window arranged on the end of the endoscope shaft inside the endoscope and an optical device which is movable in the endoscope shaft for capturing light beams and/or objects, the focusing of captured objects is improved, as the focusing optical unit has focal distances which are dependent on the viewing angle(s) of the device for capturing objects. In this case, the focusing optical unit, for example a gradient index lens, is configured such that when changing a viewing angle, for example when using a prism unit with a pivotable prism, the focal distances of the focusing optical unit are varied, whereby, depending on the viewing angle in the endoscope with an adjustable viewing direction, the focusing of the objects is facilitated in a simple manner due to the variable focal distances at different viewing angles. For example, when using at least one gradient index lens for the focusing optical unit, the focusing of the objects is achieved in a simple manner due to the variable refractive index of the gradient index lens.

As the focusing optical unit is arranged between the distal viewing window of the endoscope shaft and the movable optical device this results in the endoscope shaft having a compact character.

A focusing optical unit, for example a gradient index lens or a diffractive optical element (DOE), is a translucent element which produces a lens effect, which can be by means of a discontinuous or continuous alteration to the refractive index. In this case, the light beams are deflected and bundled at a focal point in the focusing optical unit at a predetermined viewing angle. When the viewing angle is changed, the light beams are bundled at a different focal point since, due to the focusing which is dependent on the viewing angle, the focusing optical unit has different focal distances at different viewing angles.

In this case, at least one image sensor can be arranged in the endoscope, for example at the proximal end downstream of the prism unit, wherein a prism of the prism unit arranged distally or altering a viewing angle is rotatable about an axis of rotation transversely to the longitudinal axis of the endoscope shaft. Moreover, the prism unit and the at least one image sensor can be arranged in a hermetically sealed space inside the cladding tube. Moreover, an operating element for adjusting the viewing angle of the rotatable prism can be provided, for example, on a handle of the endoscope.

The focusing optical unit can be configured such that when altering the viewing direction the focal distance of the focusing optical unit is shortened subject to an increase in the viewing angle, or the focal distance of the focusing optical unit is increased subject to a reduction in the viewing angle.

In addition, the focusing optical unit can be arranged on the viewing window and/or the focusing optical unit can be adhesively bonded to the viewing window.

An embodiment includes the focusing optical unit having a gradient index lens and/or a diffractively acting optical body, such as a film, and/or a refractively acting optical body, such as a film.

In one embodiment, the gradient index lens can be arranged on the distal viewing window of the endoscope shaft.

In one embodiment, the gradient index lens or the diffractively or refractively acting film can be adhesively bonded onto the viewing window, such as a viewing window made of sapphire.

The refractive index of a gradient index lens provided as a focusing optical unit can vary transversely to the optical axis of the lens, whereby the gradient index lens can be of compact construction and produced with a very small diameter.

Moreover, the gradient index lenses can be focusing optical units, in that the lens length is adjustable in a variable manner, wherein it is possible that the focal distance and the operating distance may be brought very close to the ends of the gradient index lenses. Moreover, in the gradient index lens, the refractive index can be designed to be different or respectively variable transversely to the optical axis of a lens. By means of the variable refractive index transversely to the optical axis a lens effect is produced so that when light beams pass through the gradient index lens the light beams are bundled or respectively focused at a focal point located in the vicinity of the gradient index lens.

By means of the arrangement of the focusing optical unit, the focal distances thereof varying depending on the viewing angle of the endoscope, between the distal viewing window and the movable optical device in the endoscope shaft, the focusing of the incident light beams from the objects and thus the optical capture of desired objects can be improved.

Moreover, in one embodiment, the optical device can be movably arranged in the direction of the longitudinal axis of the endoscope shaft and/or transversely to the longitudinal axis of the endoscope shaft. If, for example, the optical device is configured as a prism unit, the prism unit may be rotated about the longitudinal axis of the endoscope shaft. Moreover, a pivotable prism can be provided in the prism unit, said pivotable prism being pivotable about an axis which can be oriented transversely or respectively perpendicular to the longitudinal axis of the endoscope shaft.

By the pivoting of a prism, it is possible to alter the viewing direction relative to the longitudinal axis of the endoscope shaft.

The optical device can be configured as a prism unit, wherein the prism unit as a movable unit has at least one prism which is pivotable about a pivot axis.

Moreover, the optical device can be configured as an optical image sensor unit, the light beams incident from an object through the viewing window being captured thereby. The image sensor unit can be a CCD camera.

Moreover, the focusing optical unit can be configured such that the focusing optical unit has a predetermined freeform surface. As a result, it is possible that a diffractive or refractive film or respectively a diffractive optical element (DOE) is able to be produced as a focusing optical unit, wherein the film or the diffractive optical element (DOE) is arranged or respectively adhesively bonded on the inside, such as on a distal viewing window configured as a sapphire dome.

Furthermore, the focusing optical unit can be produced from plastics material. As a result, diffractive optical elements (DOE) or gradient index lenses can be produced to be of compact construction and in a simple manner.

Moreover, a focusing optical unit can be used in an endoscope, such as a video endoscope, such as for surgical investigations, wherein the endoscope or respectively video endoscope can be configured as described above.

Further features are revealed from the description of embodiments together with the claims and accompanying drawings. Embodiments may be fulfilled by individual features or a combination of several features.

### BRIEF DESCRIPTION OF THE DRAWINGS

The embodiments are described hereinafter without limiting the general inventive idea using such exemplary embodiments with reference to the drawings, wherein with regard to all of the details not described in more detail in the text, reference is expressly made to the drawings, in which:

FIG. 1 illustrates a schematic perspective view of a video endoscope,

FIG. 2 illustrates a schematic side view of a prism unit at the distal tip of an endoscope shaft,

FIG. 3 illustrates a schematic plan view of the prism unit of FIG. 2,

FIG. 4 illustrates a cross section through the distal tip of an endoscope shaft according to a further exemplary embodiment.

In the drawings in each case the same or similar elements and/or parts are provided with the same reference numerals, so that in each case a further image is dispensed with.

### DETAILED DESCRIPTION

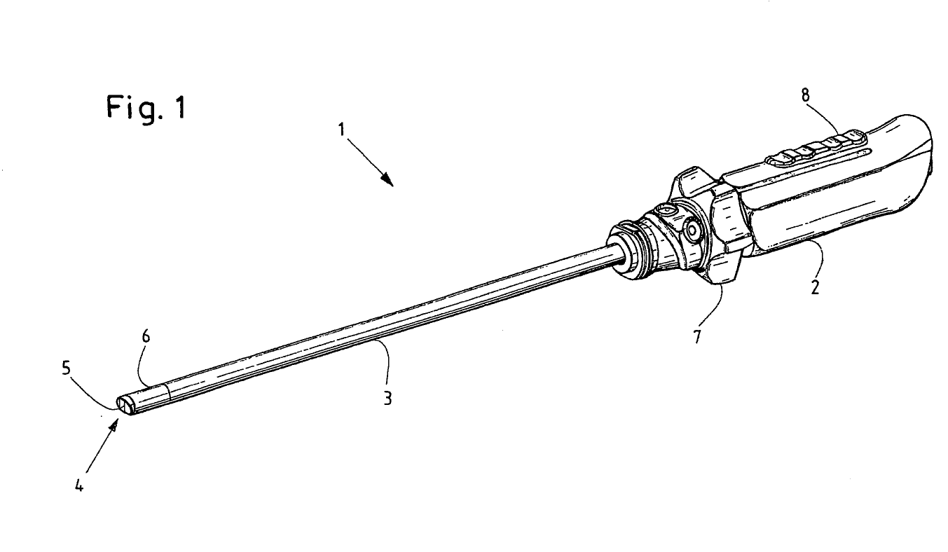

FIG. 1 shows a schematic perspective view of a video endoscope 1 having a proximal handle 2 and a rigid endoscope shaft 3. A viewing window 5 is arranged at the distal tip 4 of the endoscope shaft 3, a distal portion 6 of the endoscope shaft 3 being arranged to the rear of said viewing window, said distal portion having a prism unit, not shown, and an image sensor unit, not shown.

The viewing window 5 at the distal tip 4 is curved and of asymmetrical design. Thus the viewing window 5 is configured to assist a variable lateral viewing angle. An alteration to the viewing direction, i.e. an alteration to the azimuth angle about the longitudinal axis of the endoscope shaft 3, is effected by a rotation of the handle 2 about the central axis of rotation or respectively longitudinal axis of the endoscope shaft 3. The cladding tube of the endoscope shaft 3 is connected to the handle 2. The prism unit, not shown, is also rotated at the distal tip 4 by the rotation of the handle 2. In one embodiment, the viewing window is configured as a sapphire dome.

The handle 2 has a first operating element configured as a rotary wheel 7 and a second operating element configured as a sliding switch 8. For maintaining the horizontal position of the image shown, the rotary wheel 7 is secured during a rotation of the handle 2. As a result, the image sensor in the inside of the endoscope shaft 3 does not perform the movement therewith.

In order to alter the viewing angle, i.e. the deviation of the viewing direction from the view straight ahead, the sliding switch 8 is moved. Moving the sliding switch 8 to the distal end, for example, leads to an increase in the viewing angle, and a return of the sliding switch 8 to the proximal end in this case causes the viewing angle to be reduced to the view straight ahead. The actuation of the sliding switch 8 causes a rotation of the image sensor, in order to maintain the horizontal position of the image shown, even with a rotation of the prism units relative to one another.

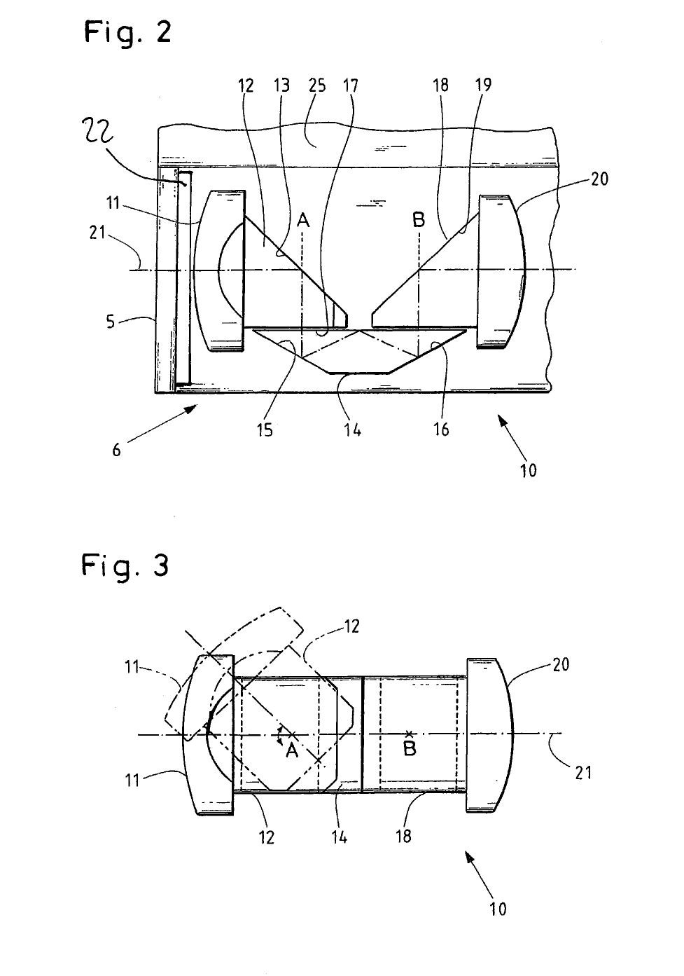

In FIG. 2, the distal end of the endoscope shaft 3 with a prism unit 10 is shown schematically from the side. On the left-hand side of the image, light of a central beam path 21 which is shown as a dashed-dotted line enters a viewing window 5 of the endoscope shaft and passes through a gradient index lens 22 arranged on the viewing window 5 into a first distal prism 12 of the prism unit 10. The gradient index lens 22 is arranged or adhesively bonded in the endoscope shaft 3 on the inside, for example by being adhesively bonded to the viewing window 5.

By means of the gradient index lens 22 which is arranged in contact with the distal viewing window 5, the incident light beams are focused due to the different refractive indices of the gradient index lens 22 which are variable, depending on the distance from the optical axis.

By means of an entry lens 11 arranged on the prism 12, the incident light beams are focused immediately downstream of the gradient index lens 22 due to the refractive index, which changes depending on the distance from the optical axis, after the passage of the light beams therethrough. The light is incident on the reflective surface 13 of the prism 12 and is reflected downwardly in the direction of a second prism 14 of the prism unit 10 and a reflective surface 15 of the second prism 14.

The reflective surface 15 of the prism 14 has an acute angle to the lower face 17 of the second prism 14 so that the central beam path is initially reflected on a central portion of the lower face 17, which is also reflective, and from there to a second reflective surface 16 of the second prism 14. This second reflective surface 16 also has an acute angle to the lower face 17, so that the central beam path in turn is reflected upwardly (axis B). Here the light is incident in a third prism 18 of the prism unit 10 with a reflective surface 19, the light of the central beam path 21 in turn being reflected centrally therethrough in a direction parallel to the longitudinal axis of the endoscope shaft 3 and emerging from the prism unit 10 through an exit lens 20.

Moreover, a part of an optical fiber bundle 25 is shown above the prism unit 10, light being conducted thereby from the proximal tip to the distal tip in order to illuminate an otherwise unilluminated field of operation.

The first prism 12 of the prism unit 10 is rotated or respectively pivoted about the perpendicular axis A, which is also denoted as the pivot axis, in order to adjust the lateral viewing angle. As a result, the reflective surface 13 of the first prism 12 and the reflective surface 15 of the fixed prism 14 of the prism unit 10 rotate relative to one another so that the horizontal position of the image, which is forwarded to the proximal end, is altered with a rotation of the first pivotable prism 12 about the axis A. This has to be compensated by a rotation of the image sensor or the image sensors.

In FIG. 3 the prism unit 10 of FIG. 2 is shown in a schematic plan view. The first prism 12 is arranged in a 0° viewing direction. The first prism 12 is pivotably mounted together with the entry lens 11 about the pivot axis A. In this case, the overlapping region between the reflective surfaces 13 of the first prism 12 and 15 of the second prism 14 is rotated. With a rotational or respectively pivoting movement of the first prism 12 the horizontal line is rotated, as is to be described below. If the prism unit 10 is arranged such that the axis of rotation A in FIG. 2 is arranged upwardly, i.e. perpendicular to the horizontal, which is an imaginary horizontal line, this horizontal line represents a line which is level with the reflective surface 13 of the prism 12. With a rotation of the first prism 12 about the axis of rotation this is independent of the angle of rotation.

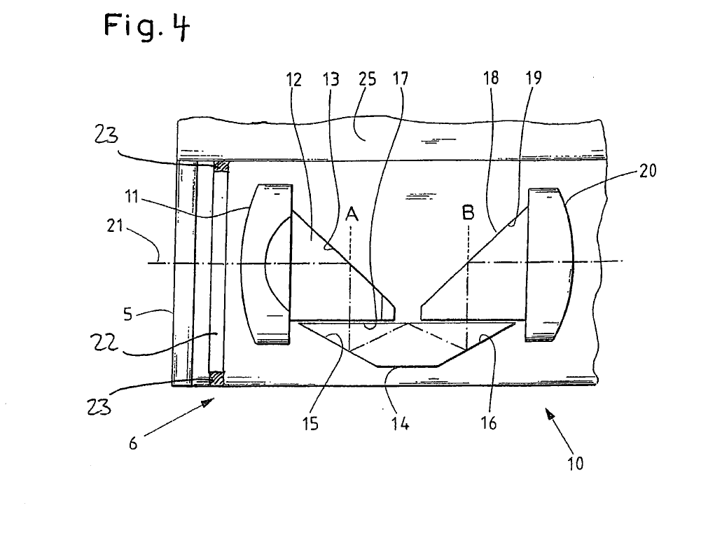

In FIG. 4, a further exemplary embodiment of the arrangement of a gradient index lens 22 between the distal viewing window 5 and the prism unit 10 is shown schematically in cross section. According to the exemplary embodiment in FIG. 4, the gradient index lens 22 is arranged in the endoscope shaft 3 between the prism unit 10 and the distal viewing window 5, wherein the gradient index lens 22 in this case is not in contact with the viewing window 5 or with the entry lens 11 of the prism 12 of the prism unit 10. For the arrangement of the gradient index lens 22 between the viewing window 5 and the prism unit 10 a mount 23 is provided so that the gradient index lens 22 is enclosed in the endoscope shaft 3.

Within the scope of the invention according to an alternative (not shown here), instead of a gradient index lens or the gradient index lens 22 it is also provided to arrange a focusing optical unit with a diffractive and/or refractive property or action, for example a diffractive film and/or refractive film, between the viewing window 5 and the prism unit 10. Moreover, corresponding combinations may be also implemented.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

### LIST OF REFERENCE NUMERALS

1 Video endoscope

2 Handle

3 Endoscope shaft

4 Distal tip

5 Viewing window

6 Distal portion

7 Rotary wheel

8 Sliding switch

9 Cladding tube

10 Prism unit

11 Entry lens

12 First prism

13 Reflective surface

14 Second prism

15, 16 Reflective surface

17 Lower face

18 Third prism

19 Reflective surface

20 Exit lens

21 Central beam path

22 Gradient index lens

23 Mount

25 Optical fiber bundle

What is claimed is:

1. An endoscope having an adjustable viewing direction at a viewing angle, the endoscope comprising:
an endoscope shaft having a longitudinal axis, wherein the endoscope shaft has a distal viewing window,
a movable optical device for capturing objects provided in the endoscope shaft, and
a focusing optical unit arranged between a distal viewing window and the movable optical device,
wherein a focal distance of the focusing optical unit is changed in dependence on the viewing angle.

2. The endoscope according to claim 1, wherein the focusing optical unit is configured such that when adjusting the viewing direction, the focal distance of the focusing optical unit is shortened subject to an increase in the viewing angle, or the focal distance of the focusing optical unit is increased subject to a reduction in the viewing angle.

3. The endoscope according to claim 1, wherein the focusing optical unit is one or more of arranged on the viewing window and adhesively bonded to the viewing window.

4. The endoscope according to claim 1, wherein the focusing optical unit has one or more of a gradient index lens, a diffractively acting optical body and a refractively acting optical body.

5. The endoscope of claim 4, wherein the diffractively acting optical body is a film.

6. The endoscope of claim 4, wherein the refractively acting optical body is a film.

7. The endoscope according to claim 1, wherein the optical device is movably arranged in the direction of one or more of the longitudinal axis of the endoscope shaft and transversely to the longitudinal axis of the endoscope shaft.

8. The endoscope according to claim 1, wherein the optical device is configured as a prism unit, wherein the prism unit has at least one prism pivotable about a pivot axis.

9. The endoscope according to claim 1, wherein the optical device is configured as an optical image sensor unit.

10. The endoscope according to claim 1, wherein the focusing optical unit has a predetermined freeform surface.

11. The endoscope according to claim 1, wherein the focusing optical unit is produced from plastics material.

12. A method for using the focusing optical unit according to claim 1 in an endoscope.
